(19) United States
(12) Patent Application Publication
(10) Pub. No.: US 2006/0011462 A1
(43) Pub. Date: Jan. 19, 2006

(54) Process for producing carboxylic acid and system for producing the same

(76) Inventors: Akira Horiguchi, Osaka (JP); Tomohide Ina, Hyogo (JP); Hiroyuki Miura, Hyogo (JP)

Correspondence Address: BIRCH STEWART KOLASCH & BIRCH, PO BOX 747, FALLS CHURCH, VA, 22040-0747, US

(21) Appl. No.: 10/528,689

(22) PCT Filed: Oct. 3, 2003
(86) PCT No.: PCT/JP03/12696

§ 371 Date: Mar. 22, 2005

(30) Foreign Application Priority Data
Oct. 8, 2002 (JP) 2002-294514

(51) Int. Cl. B01D 3/34; C07C 409/24

(52) U.S. Cl. 203/29; 203/60; 203/74; 562/6; 202/154; 202/153; 202/155

(57) ABSTRACT

In the presence of a catalytic system, an alcohol having “n” carbon atom(s) or a derivative thereof is allowed to react with carbon monoxide in a reactor 3 continuously, a higher bp catalyst component is separated from the resultant reaction mixture by a catalyst-separating column 5 to give a crude mixture, the crude mixture is fed to a higher bp component-separation column 8 to separate an overhead fraction from a bottom fraction containing at least a carboxylic acid having “n+2” carbon atoms, the overhead fraction is fed to a carboxylic acid-separating column 11, and are distilled in the presence of at least water and an ester of the carboxylic acid with the alcohol to separate a overhead fraction containing at least the ester and water from a bottom fraction containing the carboxylic acid having “n+1” carbon atoms. The overhead fraction from the carboxylic acid-separating column is fed to an aldehyde-separating column 14 to remove an overhead fraction containing an aldehyde. Such a process insures that impurities are efficiently separated from a reaction mixture by carbonylation of an alcohol, and that a carboxylic acid is purified easily at a lower cost.

### TECHNICAL FIELD

The present invention relates to a process for industrially producing a carboxylic acid such as acetic acid, in particular a process for producing a carboxylic acid by a carbonylation reaction of an alcohol (such as methanol) or a derivative thereof, and to a system for producing the same.

### BACKGROUND ART

Carboxylic acids, notably acetic acid, have been used as an ingredient of acetic acid ester compounds, acetic anhydride, terephthalic acid or others, and are one of basic chemicals being in heavy usage in various fields such as the petrochemical industry, the organic synthesis industry, the pharmaceutical and agricultural chemical industry, or the industry of polymer chemistry.

As a process for industrially producing acetic acid, various methods such as oxidation of acetaldehyde and direct oxidation of a hydrocarbon (e.g., petroleum naphtha, butane) have been known. Among others, a method currently universally adopted for industrially producing acetic acid is a method of producing acetic acid by continuously allowing methanol to react with carbon monoxide to carbonylate methanol [Japanese Patent Publication No. 3334/1972 (JP-47-3334B)].

Regarding the foregoing production method of acetic acid by carbonylation of methanol, New Petrochemical Process (Japan Petroleum Institute) p. 316 (1986) describes purification of acetic acid by the following four distillation steps (1) to (4):
(1) in a lower-boiling component-separation column, separating a fraction having a lower-boiling point through the overhead of the column, and in parallel separating a higher boiling point fraction containing a catalyst through the bottom of the column, and returning the bottom fraction to a reactor,
(2) in a dehydration column, separating moisture not removed by the lower-boiling component-separation column through the overhead of the dehydration column, and returning the moisture to the reactor,
(3) in a distillation column for obtaining acetic acid, separating propionic acid being a component having a higher-boiling point through the bottom of the distillation column, and
(4) in a purification column, separating slight amounts of a lower-boiling point fraction and higher-boiling point fraction through the overhead and bottom of the purification column, respectively.

In a binary system of acetic acid and water, however, it is difficult to separate acetic acid from water because of a low relative volatility between water and acetic acid based on the relation of vapor-liquid equilibrium, and therefore, it is necessary to increase the number of plates or to enhance the reflux ratio of a distillation column in order to separate acetic acid from water efficiently. In particular, industrial production of acetic acid needs removal of water from a crude reaction solution in a purification step. However, it is difficult to separate of water from acetic acid so that equipment expenses and energy cost are significantly increased due to increase of the number of plates or enhancement of the reflux ratio.

Moreover, in the case producing acetic acid by carbonylation of methanol, since the reaction needs water, the crude reaction solution contains water. In order to obtain acetic acid as a finished product, water must be removed so that the moisture content becomes not more than a given concentration. Generally, as described above, water is removed with the use of the dehydration column. However, an excess amount of acetic acid is distilled off along with water, and the resultant mixture of acetic acid and water is recycled to a reactor. Such a method causes significantly large energy loss because the excess amount of acetic acid circulates through the system.

Japanese Patent Publication No. 30093/1982 (JP-57-30093B) proposes a method for separating acetic acid which comprises adding methyl acetate as a third component in a dehydration column, and allowing methyl acetate to azeotrope with water. In order to add the third component, however, extra equipment and control are necessary, in addition there is also a possibility that the third component is contaminated in acetic acid as a finished product.

Moreover, acetaldehyde and/or propionic acid are contained in a reaction mixture obtained by carbonylation of methanol. Acetaldehyde in itself causes impairment of quality of acetic acid. In addition, circulation of acetaldehyde through the system not only results in concentration but also forms a compound having a higher-boiling point, and higher-boiling impurities are generated, whose boiling point is close to that of acetic acid as the finished product. The contamination of the impurities in the product further causes deterioration in quality of the finished product. Moreover, the contamination of the foregoing propionic acid in acetic acid as the finished product effects deterioration in quality of the subsequent product.

Regarding a method for removing a halide contained in a carboxylic acid in a ppb order, Japanese Patent Application Laid-Open No. 5367/1971 (JP-46-5367B) discloses that a halogen-free carboxylic acid is obtained by removing higher-boiling impurities in a first distillation column and removing lower-boiling impurities containing a halide in a second distillation column to purify a carboxylic acid as a product. However, this literature fails to disclose a concrete process for purifying a crude reaction solution containing additional other impurities in addition to a halide.

It is therefore an object of the present invention to provide a process and system for efficiently separating impurities from a reaction mixture obtained by a carbonylation reaction of an alcohol (in particular methanol) to produce a purified carboxylic acid (in particular acetic acid) easily and efficiently.

It is another object of the present invention to provide a process and system which insures production of a carboxylic acid (i.e., a purified carboxylic acid) with removing water without circulating an excess amount of a carboxylic acid (in particular acetic acid) through a reaction system.

It is still another object of the present invention to provide a process and system which insures production of a highly purified carboxylic acid (in particular acetic acid) without addition of an azeotropic component.

It is a further object of the present invention to provide a process and system which insures production of a highly purified carboxylic acid (in particular acetic acid) at high energy efficiency.

### DISCLOSURE OF INVENTION

The inventors of the present invention made intensive studies to achieve the above objects and finally found that beforehand removal (or elimination) of a bottom (or higher boiling point) fraction (e.g., a carboxylic acid having “n+2” carbon atoms, a higher-boiling point catalytic component) from a reaction product obtained by carbonylation of an alcohol having “n” carbon atom(s) insures utilization of water and an ester of the alcohol with a carboxylic acid having “n+1” carbon atoms generated in the reaction system, as azeotropic solvents, efficient purification of the carboxylic acid having “n+1” carbon atoms at a high energy efficiency, and thereby reducing significantly the production cost. The present invention was accomplished based on the above findings.

That is, the present invention includes a process for producing a carboxylic acid comprising
allowing an alcohol having “n” carbon atom(s) or a derivative thereof to react with carbon monoxide continuously in the presence of a catalytic system, and purifying the resultant reaction mixture to give a purified carboxylic acid having “n+1” carbon atoms, wherein a higher-boiling point (or higher bp) catalyst component is separated from the reaction mixture to give a crude mixture containing at least a carboxylic acid having “n+2” carbon atoms, a carboxylic acid having “n+1” carbon atoms, an ester of the carboxylic acid having “n+1” carbon atoms with the alcohol, and water; the crude mixture is fed to a higher-boiling point (or higher bp) component-separation column, and is separated into a bottom fraction and an overhead fraction, the bottom fraction contains at least the carboxylic acid having “n+2” carbon atoms, and the overhead fraction contains at least the carboxylic acid having “n+1” carbon atoms, the ester of the carboxylic acid having “n+1” carbon atoms with the alcohol, and water; and the overhead fraction from the higher bp component-separation column is separated by a carboxylic acid-separating column into a bottom fraction and an overhead fraction, the bottom fraction contains the carboxylic acid having “n+1” carbon atoms, and the overhead fraction contains at least the ester and water. The reaction mixture may contain water in a proportion of not more than 20% by weight.

In the production process of the present invention, the crude mixture may further contain an aldehyde having “n+1” carbon atoms, and the crude mixture may be fed to the higher bp component-separation column. In the production process, the crude mixture containing the carboxylic acid having “n+2” carbon atoms, an aldehyde having “n+1” carbon atoms, the carboxylic acid having “n+1” carbon atoms, the ester of the carboxylic acid having “n+1” carbon atoms with the alcohol and water may be fed to the higher bp component-separation column, and may be separated into the bottom fraction and the overhead fraction, the bottom fraction contains the carboxylic acid having “n+2” carbon atoms, and the overhead fraction contains the aldehyde having “n+1” carbon atoms, the carboxylic acid having “n+1” carbon atoms, the ester of the carboxylic acid having “n+1” carbon atoms with the alcohol, and water; the overhead fraction from the higher bp component-separation column may be separated by the carboxylic acid-separating column into the bottom fraction and the overhead fraction, the bottom fraction contains the carboxylic acid having “n+1” carbon atoms, and the overhead fraction contains at least the aldehyde, the ester and water; the overhead fraction from the carboxylic acid-separating column may be separated by an aldehyde-separating column into an overhead fraction and a bottom fraction, the overhead fraction contains the aldehyde, and the bottom fraction contains at least the ester and water; and the bottom fraction from the aldehyde-separating column may be recycled to the reaction system.

The catalytic system may comprise a catalyst containing a metal element of the Group 8 of the Periodic Table of Elements, and an alkyl halide (and if necessary an alkali metal halide); distillation in the carboxylic acid-separating column may be carried out in the presence of the ester of the carboxylic acid having “n+1” carbon atoms with the alcohol, the alkyl halide and water for separating the bottom fraction from the overhead fraction, the bottom fraction contains the carboxylic acid having “n+1” carbon atoms, and the overhead fraction contains water, the alkyl halide and the ester; the overhead fraction from the carboxylic acid-separating column may be separated by the aldehyde-separating column into the overhead fraction and the bottom fraction, the overhead fraction contains the aldehyde, and the bottom fraction contains water, the alkyl halide and the ester; and the bottom fraction from the aldehyde-separating column may be recycled to the reaction system.

Moreover, in the production process of the present invention, the crude mixture in which at least an aldehyde having “n+1” carbon atoms has been removed may be fed to the higher bp component-separation column. The higher bp catalyst component may be separated from the reaction mixture to give a crude mixture, and the resultant crude mixture may be fed to a lower-boiling point (or lower bp) component-separation column, and may be separated into the overhead fraction and the bottom fraction, the overhead fraction contains at least an aldehyde having “n” carbon atom(s), and the bottom fraction contains at least the carboxylic acid having “n+2” carbon atoms; the bottom fraction from the lower bp component-separation column may be separated by the higher bp component-separation column into the bottom fraction and the overhead fraction, the bottom fraction contains the carboxylic acid having “n+2” carbon atoms, and the overhead fraction contains at least the carboxylic acid having “n+1” carbon atoms, the ester of the carboxylic acid having “n+1” carbon atoms with the alcohol, and water; and the overhead fraction from the higher bp component-separation column may be separated by the carboxylic acid-separating column into the bottom fraction containing the carboxylic acid having “n+1” carbon atoms and the overhead fraction containing at least the ester and water. Distillation in the carboxylic acid-separating column may be carried out in the presence of at least the ester and water to separate the bottom fraction from the overhead fraction.

The catalytic system may comprise a catalyst containing a metal element of the Group 8 of the Periodic Table of Elements, and an alkyl halide (and if necessary an alkali metal halide); distillation in the carboxylic acid-separating column may be carried out in the presence of the ester, the alkyl halide and water to give the bottom fraction containing the carboxylic acid having “n+1” carbon atoms, and the overhead fraction containing at least the ester, the alkyl halide and water.

The overhead fraction separated by the carboxylic acid-separating column may be recycled to the reaction system. Moreover, the overhead fraction separated by the lower bp component-separation column may be further fed to an aldehyde-separating column to separate an overhead fraction containing an aldehyde having “n+1” carbon atoms to give a bottom fraction containing at least the ester and water; and the bottom fraction may be recycled to the reaction system.

According to the present invention, in a process which comprises allowing at least one member selected from the group consisting of methanol, methyl acetate and dimethyl ether to react with carbon monoxide continuously in the presence of the catalytic system, and purifying the resultant reaction mixture to produce a purified acetic acid, the higher bp catalyst component may be separated from the reaction mixture to give the crude mixture; the crude mixture may be fed to the higher bp component-separation column, and may be separated into the bottom fraction and the overhead fraction, the bottom fraction contains at least propionic acid, and the overhead fraction contains at least acetic acid, methyl acetate and water; and the overhead fraction from the higher bp component-separation column may be fed to the carboxylic acid-separating column to distill the fraction in the presence of at least the methyl acetate, and may be separated into the bottom fraction and the overhead fraction, the bottom fraction contains the acetic acid, and the overhead fraction contains at least the methyl acetate and water.

The catalytic system may comprise a catalyst containing a rhodium catalyst, an alkali metal iodide and methyl iodide; the crude mixture may be separated by the higher bp component-separation column into the bottom fraction and the overhead fraction, the bottom fraction contains at least propionic acid, and the overhead fraction contains acetic acid, methyl acetate, methyl iodide and water; and the overhead fraction from the higher bp component-separation column may be distilled by the carboxylic acid-separating column in the presence of the methyl acetate and methyl iodide, and may be separated into the bottom fraction and the overhead fraction, the bottom fraction contains the acetic acid, and the overhead fraction contains the methyl acetate, methyl iodide and water.

The present invention also discloses a system corresponding to the above-mentioned production process. That is, the production system of the present invention comprises a reaction system for allowing an alcohol having “n” carbon atom(s) or a derivative thereof to react with carbon monoxide continuously in the presence of a catalytic system; a catalyst-separating column for separating a higher bp catalyst component from a reaction mixture generated in the reaction system; a higher bp component-separation column for separating a crude mixture obtained by a separation in the catalyst-separating column and containing at least a carboxylic acid having “n+2” carbon atoms, a carboxylic acid having “n+1” carbon atoms, an ester of the carboxylic acid having “n+1” carbon atoms with the alcohol, and water, into a bottom fraction and an overhead fraction, wherein the bottom fraction contains at least the carboxylic acid having “n+2” carbon atoms, and the overhead fraction contains at least the carboxylic acid having “n+1” carbon atoms, the ester of the carboxylic acid having “n+1” carbon atoms with the alcohol, and water; and a carboxylic acid-separating column for separating the overhead fraction separated by the higher bp component-separation column into a bottom fraction and an overhead fraction, wherein the bottom fraction contains the carboxylic acid having “n+1” carbon atoms, and the overhead fraction contains at least the ester and water.

### BRIEF DESCRIPTION OF DRAWINGS

FIG. 1 shows a flow diagram for illustrating an embodiment of a production (purification) process of a carboxylic acid of the present invention.

FIG. 2 shows a flow diagram for illustrating another embodiment of a production (purification) process of a carboxylic acid of the present invention.

FIG. 3 shows a flow diagram for illustrating still another embodiment of a production (purification) process of a carboxylic acid of the present invention.

FIG. 4 shows a flow diagram for illustrating a purification process of a carboxylic acid of Comparative Example 1.

### DETAILED DESCRIPTION OF THE INVENTION

The present invention shall now be described in detail with reference if necessary to the attached drawings.

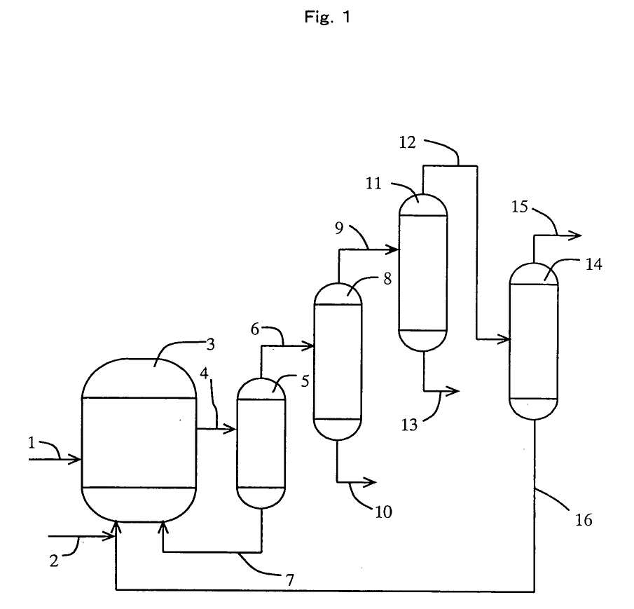

FIG. 1 is a flow diagram for explaining a production process of a carboxylic acid of the present invention.

This embodiment shows a production process for producing acetic acid (purified acetic acid) from a reaction mixture formed by a continuous carbonylation reaction of methanol and carbon monoxide in the presence of a carbonylation catalytic system composed of a rhodium catalyst and a co-catalyst (lithium iodide and methyl iodide).

The process comprises a reactor 3 for carrying out the above-mentioned carbonylation reaction of methanol; a distillation column (or catalyst-separating column) 5 for mainly separating the rhodium catalyst and lithium iodide from the reaction mixture containing acetic acid generated by the reaction; a higher-boiling point (or higher bp) component-separation column (or nonvolatile component-separation column) 8 for removing propionic acid; a carboxylic acid-separating column 11 for separating a fraction containing at least acetaldehyde from a fraction containing acetic acid; and an aldehyde-separating column 14 for removing acetaldehyde from the fraction containing at least acetaldehyde separated by the carboxylic acid-separating column 11. In this specification, the term “boiling point” is sometimes referred to as “bp”.

In more detail, the reactor 3 constitutes a liquid-phase reaction system containing a carbonylation catalytic system [a catalytic system composed of a main catalyst component (such as a rhodium catalyst) and a co-catalyst (such as lithium iodide and methyl iodide)]. In such a reactor 3, with continuous feeding of methanol as a liquid component at a predetermined rate via a feed line 2, carbon monoxide as a gaseous reaction component is directly and continuously fed via a feed line 1. Since such a liquid-phase reaction system is an exothermic reaction system that accompanies generation of heat, the reactor 3 may comprise a heat-removing unit or cooling unit (e.g., jacket) for controlling a reaction temperature.

The reaction mixture (or crude reaction solution) formed in the reactor 3 comprises, lower-boiling impurities having a boiling point lower than that of acetic acid (e.g., acetaldehyde being a precursor of acetic acid) and higher-boiling impurities having a boiling point higher than that of acetic acid (e.g., propionic acid) as impurities, in addition to metal catalyst components (a rhodium catalyst, and lithium iodide as a co-catalyst), acetic acid, methyl iodide as a co-catalyst, methyl acetate that is a reaction product of acetic acid with methanol, water, or others.

In order to purify acetic acid from such a reaction mixture, with withdrawing a fraction of the reaction mixture from the reactor 3 continuously, the withdrawn reaction mixture is fed to the catalyst-separating column 5 through a feed line 4. In the catalyst-separating column 5, a catalyst component having a higher bp (e.g., a metal-containing catalyst component such as the rhodium catalyst and lithium iodide) is withdrawn from the column bottom to separate from the reaction mixture. The higher bp catalyst component (or nonvolatile catalyst component) is a reusable fraction by recycling, and thus after separation with the catalyst-separating column 5 the fraction is recycled to the reaction system (reactor 3) through a first recycle line 7.

The overhead fraction (or lower bp fraction or stream) which is distilled out from the overhead of the catalyst-separating column 5 and contains acetic acid is fed to the higher bp component-separation column 8 through a feed line 6. In the higher bp component-separation column 8, a bottom fraction (or higher bp fraction or stream) containing at least propionic acid is separated from the column bottom through a bottom line 10. Propionic acid can be relatively easily separated from acetic acid by utilizing difference between the both in boiling point. In the higher bp component-separation column 8, to adjust the overhead temperature (or the column bottom temperature), the overhead pressure sets up in a range of about 10 to 1,000 kPa as an absolute pressure.

The overhead fraction which is distilled out from the overhead of the higher bp component-separation column 8 and contains acetic acid is fed to the carboxylic acid-separating column 11 through a feed line 9. In the carboxylic acid-separating column 11, an overhead fraction containing at least acetaldehyde is separated from the overhead, purified acetic acid can be separated and recovered from the column bottom through a bottom line 13 as a bottom (or nonvolatile) fraction.

The overhead fraction separated by the carboxylic acid-separating column 11 comprises acetaldehyde, and in addition useful components (methyl iodide of the co-catalyst, methyl acetate that is a reaction product of acetic acid with methanol, and water). In order to remove acetaldehyde among these components and to recycle the useful components to the reaction system, the overhead fraction is further fed to an aldehyde-separating column 14 through a feed line 12. Incidentally, acetic acid can be separated from acetaldehyde based on difference between the both in boiling point. Thus, in the carboxylic acid-separating column 11 acetic acid can be efficiently separated from the overhead fraction containing acetaldehyde. In particular, since methyl acetate and methyl iodide act as azeotropic components relative to water, acetic acid can be highly purified. In the carboxylic acid-separating column 11, to adjust the overhead temperature (or the column bottom temperature), the overhead pressure sets up in a range of about 10 to 1,000 kPa as an absolute pressure.

In the aldehyde-separating column 14, the overhead fraction containing acetaldehyde is separated through a distillation line 15 from the column overhead, and the bottom fraction containing the useful component (or fraction) is separated from the column bottom.

The bottom fraction separated by the aldehyde-separating column 14 usually contains water, methyl iodide being a co-catalyst, methyl acetate that is a reaction product of acetic acid with methanol, and others. In order to utilize these components as a catalyst or reaction component effectively, the bottom fraction is recycled through a second recycle line 16 to the reaction system, and is converged in methanol fed from the feed line 2 for feeding the reactor 3.

Incidentally, in the aldehyde-separating column 14, to adjust the overhead temperature, the overhead pressure sets up in a range of about 10 to 1,000 kPa as an absolute pressure.

Since such a process insures that methyl acetate and/or methyl iodide may coexist with water in the carboxylic acid-separating column 11, not only acetaldehyde but also water that has difficulty separating from acetic acid may be efficiently removed by azeotrope of the foregoing methyl iodide or methyl acetate with water. Therefore, acetic acid may be separated from water without increasing the number of plates of the distillation column or enhancing the reflux ratio. Moreover, acetic acid can be efficiently recovered as a finished product without circulating a large amount of acetic acid within the reaction system. Further, in the aldehyde-separating column, since the vapor pressure of acetaldehyde is high in the overhead fraction to be fed, acetaldehyde may be accurately separated from the useful component (or fraction) or the bottom fraction, and deterioration in purification efficiency of acetic acid may be inhibited by circulating of acetaldehyde within the reaction system. Moreover, methyl iodide, water or the like separated by the aldehyde-separating column may be effectively utilized by recycling to the reaction system. In addition, by recycling water to the reaction system, the catalytic system in the reaction system may be stabilized. Therefore, impurities may be separated efficiently at high-energy efficiency, the vapor amount to be used for heating of the higher bp component-separation column to the aldehyde-separating column may be drastically reduced, and cost of equipment may also be cut down.

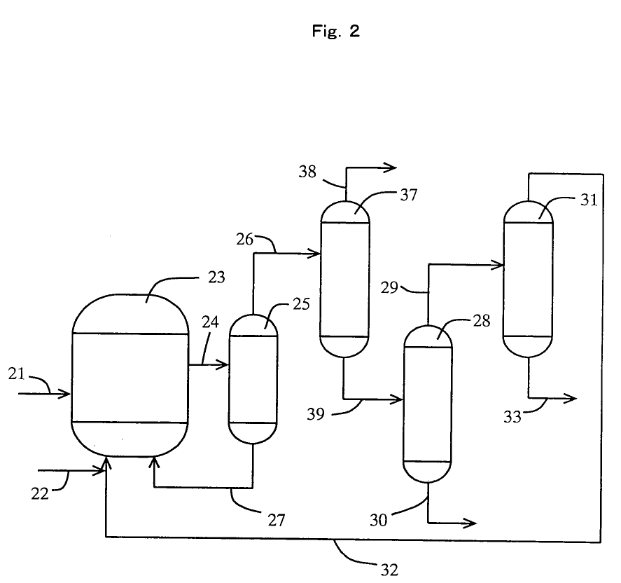

FIG. 2 is a flow diagram for illustrating another embodiment of a production process of acetic acid of the present invention.

This embodiment shows a process which comprises feeding the overhead fraction separated by the catalyst-separating column in the embodiment of FIG. 1 to a lower bp component-separation column to separate a bottom fraction from an overhead fraction containing at least acetaldehyde through the lower bp component-separation column, and then feeding the bottom fraction separated by the lower bp component-separation column to a higher bp component-separation column. Such a process is useful as a system for highly removing an aldehyde from the objective carboxylic acid.

The process comprises a reactor 23 for carrying out the carbonylation reaction of the above-mentioned methanol; a catalyst-separating column 25 for mainly separating a higher bp catalyst fraction (or component) (a rhodium catalyst and lithium iodide) from the reaction mixture containing acetic acid generated by the reaction; a lower bp component-separation column 37 for separating acetaldehyde; a higher bp component-separation column 28 for removing propionic acid; and a carboxylic acid-separating column 31 for separating water. Incidentally, in the same manner as in the embodiment of FIG. 1, carbon monoxide and methanol may be fed to the reactor through feed lines 21 and 22, respectively.

In this embodiment, similar to the embodiment of FIG. 1, the overhead fraction which is distilled off from the catalyst-separating column 25 and contains acetic acid is fed to the lower bp component-separation column 37 through a feed line 26. In the lower bp component-separation column 37, an overhead fraction containing at least acetaldehyde is separated through a distillation line 38 from the column overhead. Incidentally, since acetaldehyde can be easily separated from acetic acid, in the lower bp component-separation column 37 acetaldehyde can be efficiently discharged or distilled off as an overhead fraction out of the system.

In the lower bp component-separation column 37, to adjust the overhead temperature, the overhead pressure sets up in a range of about 10 to 1,000 kPa as an absolute pressure. Incidentally, in the case where the overhead temperature of the lower bp component-separation column is high, not only acetaldehyde but also methyl iodide as a co-catalyst, methyl acetate that is a reaction product of acetic acid with methanol, water, acetic acid, and others are sometimes distilled off as an overhead fraction. In such a case, acetaldehyde may be further removed from the distillate for recycling the residual fraction to the reaction system.

The bottom fraction which is withdrawn from the column bottom of the lower bp component-separation column 37 and contains acetic acid is fed to a higher bp component-separation column 28 through a feed line 39. In the higher bp component-separation column 28, a bottom fraction containing at least propionic acid is separated from the column bottom through a bottom line 30. Propionic acid may be relatively easily separated from acetic acid using difference between the both in boiling point. In the higher bp component-separation column 28, to adjust the overhead temperature (or the column bottom temperature), the overhead pressure sets up in a range of about 10 to 1,000 kPa as an absolute pressure.

The overhead fraction (liquid or gas) which is distilled off from the overhead of the higher bp component-separation column 28 and contains acetic acid is further fed to a carboxylic acid-separating column 31 through a feed line 29. In the carboxylic acid-separating column 31, the overhead fraction containing at least water is separated from the overhead, and purified acetic acid may be separated as a bottom fraction through a bottom line 33 from the bottom of the column. In the carboxylic acid-separating column 31, to adjust the overhead temperature (or the column bottom temperature), the overhead pressure sets up in a range of about 10 to 1,000 kPa as an absolute pressure.

The overhead fraction distilled off from the overhead of the carboxylic acid-separating column 31 comprise water, in addition, methyl iodide as a co-catalyst, methyl acetate that is a reaction product of acetic acid with methanol, or others. In order to utilize these components as a catalyst or reaction component effectively, the overhead fraction is recycled to the reaction system through a second recycle line 32, and is converged in methanol fed from a feed line 22 for feeding to the reactor 23. Thus, recycle of water can stabilize the catalytic system in the reaction system.

According to such a process, in the carboxylic acid-separating column 31, methyl acetate or methyl iodide coexists with water to allow to azeotrope with water efficiently so that water may be removed. Therefore, acetic acid may be separated from water without increasing the number of plates of the distillation column or enhancing the reflux ratio. Moreover, acetic acid may be recovered as a finished product without circulating a large amount of acetic acid within the reaction system. As the result, impurities may be separated efficiently at high energy efficiency, the vapor amount to be used for heating the lower bp component-separation column, the higher bp component-separation column and the carboxylic acid-separating column may be drastically reduced, and cost of equipment may be also cut down.

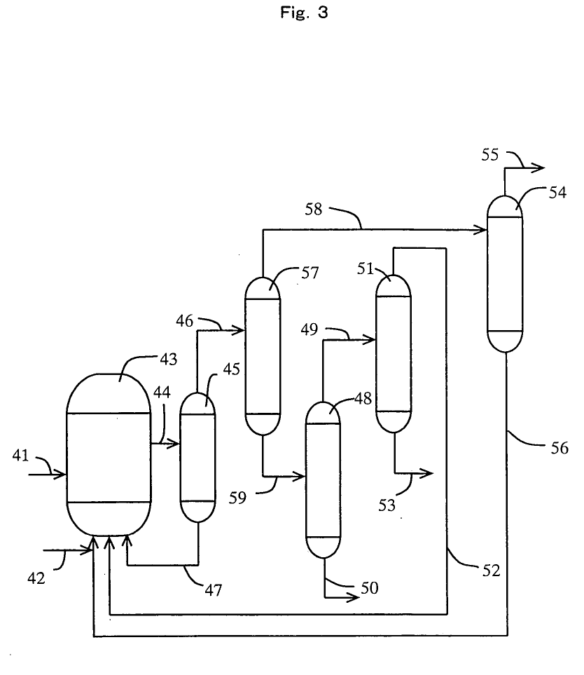

FIG. 3 is a flow diagram for illustrating still another embodiment of a production process of the present invention.

This embodiment shows a process useful for a system in which an overhead fraction distilled from the lower bp component-separation column comprises acetaldehyde, in addition methyl iodide as a co-catalyst, and in some cases further comprises methyl acetate, water, and others in the embodiment of FIG. 2.

The process comprises a reactor 43 for carrying out a carbonylation reaction of methanol; a catalyst-separating column 45 for mainly separating higher bp catalyst component (a rhodium catalyst and lithium iodide) from the reaction mixture containing acetic acid formed by the reaction; a lower bp component-separation column 57 for separating at least acetaldehyde and methyl iodide as a co-catalyst; a higher bp component-separation column 48 for removing propionic acid; a carboxylic acid-separating column 51 for separating at least water; and an aldehyde-separating column 54 for removing acetaldehyde from an overhead fraction containing acetaldehyde and methyl iodide which are separated by the lower bp component-separation column 57. Incidentally, in the same manner as in the embodiment of FIG. 2, carbon monoxide and methanol may be fed to the reactor through feed lines 41 and 42, respectively.

In this embodiment, the overhead fraction which is distilled off from the overhead of the catalyst-separating column 45 and contains acetic acid is fed to the lower bp component-separation column 57 through a feed line 46 similar to the embodiment of FIG. 2. In the lower bp component-separation column 57, an overhead fraction containing at least acetaldehyde is separated from the overhead. As described above, in the case where the distillation temperature (overhead temperature) of the lower bp component-separation column is high, the overhead fraction in the lower bp component-separation column also comprises methyl iodide, and methyl acetate, water, acetic acid, or others in addition to acetaldehyde. Components such as methyl iodide, methyl acetate, water and acetic acid may be recycled to the reaction system (reactor 43), however, acetaldehyde deteriorates purification efficiency of acetic acid. Therefore, acetaldehyde is removed by feeding the overhead fraction distilled off from the overhead of the lower bp component-separation column 57 to the aldehyde-separating column 54 through a feed line 58.

In the lower bp component-separation column 57, to adjust the overhead temperature, the overhead pressure sets up in a range of about 10 to 1,000 kPa as an absolute pressure.

According to this embodiment, in the lower bp component-separation column 57, since acetaldehyde is separated with a high degree of accuracy by enhancing the overhead temperature, the load in the higher bp component-separation column and carboxylic acid-separating column is relievable, and therefore impurities are removable efficiently.

To the higher bp component-separation column 48 the bottom fraction (i.e., higher bp fraction) withdrawn from the column bottom of the lower bp component-separation column 57 is fed, and is separated into the two fractions, a bottom fraction which is withdrawn from the bottom and contains at least propionic acid, and an overhead fraction which is distilled off from the overhead and contains acetic acid, and the overhead fraction is fed to the carboxylic acid-separating column 51. In the higher bp component-separation column 48, to adjust the overhead temperature (or the column bottom temperature), the overhead pressure sets up in a range of about 10 to 1,000 kPa as an absolute pressure.

To the carboxylic acid-separating column 51 the overhead fraction (i.e., lower bp fraction) distilled off from the overhead of the higher bp component-separation column 48 is fed, and is separated into the two fractions, an overhead fraction which is distilled off from the overhead and contains at least water, and a bottom fraction which is withdrawn from the column bottom and contains purified acetic acid. The overhead fraction separated from the overhead of the carboxylic acid-separating column 51 comprises water, methyl iodide as a co-catalyst, methyl acetate and others, and is recycled to the reaction system in the same manner as in the embodiment of FIG. 2. Incidentally, in the carboxylic acid-separating column 51, to adjust the overhead temperature (or the column bottom temperature), the overhead pressure sets up in a range of about 10 to 1,000 kPa as an absolute pressure.

The overhead fraction distilled off from the overhead of the lower bp component-separation column 57 is fed to the aldehyde-separating column 54 through the feed line 58. In the aldehyde-separating column 54, the overhead fraction containing acetaldehyde is distilled and removed from the overhead of the column through a distillation line 55, and the bottom fraction is separated from the bottom of the column. The bottom fraction separated from the bottom of the column comprises methyl iodide, water, methyl acetate, and others. In order to utilize these components effectively, the bottom fraction is recycled to the reaction system (reactor 43) through a third recycle line 56, and is converged in methanol fed from a feed line 42 for feeding the reactor 43. In the aldehyde-separating column, to adjust the overhead temperature, the overhead pressure sets up in a range of about 10 to 1,000 kPa as an absolute pressure.

Such a process ensures accurate separation of acetaldehyde in the lower bp component-separation column 57, and it can be inhibited that purification efficiency of acetic acid deteriorates due to circulation of acetaldehyde through the reaction system. Moreover, since acetaldehyde can be separated with a high degree of accuracy in the lower bp component-separation column 57, in the higher bp component-separation column 48 and carboxylic acid-separating column 51 the load is relievable, and impurities are separable efficiently.

Further, in the carboxylic acid-separating column 51, in the same manner as in the embodiment of FIG. 2, methyl acetate or methyl iodide coexists with water to allow to azeotrope with water efficiently so that water may be removed to be separated from acetic acid. Moreover, acetic acid can be collected as a finished product without circulating a large amount of acetic acid through the reaction system.

Moreover, by recycling components such as methyl iodide and water separated by the carboxylic acid-separating column 51 and the aldehyde-separating column 54 to the reaction system, these components may be utilized effectively, and in addition, by recycling water to the reaction system, the catalytic system in the reaction system may be stabilized.

Therefore, impurities may be separated efficiently at high energy efficiency, the vapor amount to be used for heating of the lower bp component-separation column to the aldehyde-separating column may be drastically reduced, and cost of equipment may be also cut down.

The production process of the present invention comprises a carbonylation reaction (i.e., a reaction step) for forming a carboxylic acid and a purification process of the carboxylic acid (including a step for separating a higher bp catalyst component, and a step for purifying the carboxylic acid), and is applicable for carbonylation reactions of various alcohols or derivatives thereof, not being limited to the foregoing carbonylation reaction of methanol.

(Carbonylation Reaction)

In a carbonylation reaction, an alcohol or a derivative thereof (reactive derivative) is carbonylated with carbon monoxide. As the alcohol to be used in the carbonylation reaction, there may be exemplified an alcohol having “n” carbon atom(s), for example, an aliphatic alcohol [e.g., an alkanol (e.g., a C₁₋₁₀alkanol) such as methanol, ethanol, propanol, isopropanol, butanol, pentanol, or hexanol], an alicyclic alcohol [e.g., a cycloalkanol (e.g., a C₃₋₁₀ cycloalkanol) such as cyclohexanol or cyclooctanol], an aromatic alcohol [an aryl alcohol (e.g., a C₆₋₁₀aryl alcohol (such as a phenol compound)) such as phenol; an aralkyl alcohol (e.g., a C₆₋₁₀aryl-C₁₋₄alkanol) such as benzyl alcohol or phenethyl alcohol], or others. The number “n” of carbon atom is about 1 to 14, preferably about 1 to 10, and more preferably about 1 to 6. Among the foregoing alcohols, an aliphatic alcohol is preferred. The number “n” of carbon atom in the aliphatic alcohol is, for example, about 1 to 6, preferably about 1 to 4, and in particular about 1 to 3.

Among the alcohol derivatives, an ester compound includes an ester of a carboxylic acid to be formed with a raw alcohol, for example, a C₁₋₆alkyl ester of a C₂₋₆carboxylic acid such as methyl acetate or ethyl propionate, or others. An ether compound includes an ether corresponding to the raw alcohol, for example, a diC₁₋₆alkyl ether such as methyl ether, ethyl ether, propyl ether, isopropyl ether or butyl ether, or the like. If necessary, as an alcohol, there may be used a polyhydric alcohol, for example, an alkylene glycol such as ethylene glycol, propylene glycol or butanediol, or a derivative thereof (e.g., an ester, a halide, an ether).

The alcohol or a derivative thereof may be used singly or in combination.

In the preferred liquid-phase reaction system, an alcohol having “n” carbon atom(s) as a liquid reaction component, preferably a C₁₋₄alcohol or a derivative thereof (e.g., methanol, methyl acetate, methyl iodide, dimethyl ether) may be used to obtain a carboxylic acid having “n+1” carbon atoms or a derivative thereof (e.g., a carboxylic anhydride). In particular, the following reaction system is preferred: a reaction system in which at least one member selected from the group consisting of methanol, methyl acetate, and dimethyl ether (particularly at least methanol) is allowed to react with carbon monoxide in the presence of a carbonylation catalyst or a catalytic system in a liquid-phase reaction system, to produce acetic acid or a derivative thereof.

Incidentally, the alcohol or a derivative thereof may be directly fed to the reaction system without going through the recycle line. Moreover, an alcohol or a derivative thereof distilled off from a purification step (for example, a carboxylic acid-separating column shown in FIG. 2, or an aldehyde-separating column shown in FIGS. 1 and 3) may be usually fed to a reactor through a recycle line.

The liquid-phase reaction can be carried out in the presence of various catalytic systems, not being limited to the foregoing catalytic system. The catalytic system usually comprises a carbonylation catalyst, and a co-catalyst or accelerator.

As the carbonylation catalyst, there may be usually employed a catalyst having a high boiling point, e.g., a metal catalyst. Such a catalyst includes a transition metal catalyst, in particular a metal catalyst containing a metal element of the Group 8 of the Periodic Table of Elements, for example, a cobalt catalyst, a rhodium catalyst, an iridium catalyst, or others. The catalyst may be a simple metal, or may be used in the form of a metal oxide (including a complex metal oxide), an metal hydroxide, a metal halide (e.g., a chloride, a bromide, a iodide), a metal carboxylate (e.g., an acetate), a metal salt of an inorganic acid (e.g., a sulfate, a nitrate, a phosphate), a metal complex or others. Such a metal catalyst may be used singly or in combination.

The preferred metal catalyst includes a rhodium catalyst and an iridium catalyst (in particular a rhodium catalyst). Incidentally, rhodium usually exists as a complex in a reaction solution, and in the case using a rhodium catalyst, the catalyst is not particularly limited as far as the catalyst can change into a complex in a reaction solution, and may be used in various forms. As such a rhodium catalyst, a rhodium halide (such as bromide or iodide) is particularly preferred. Moreover, the catalyst may be stabilized in a reaction solution by adding a salt of a halide (e.g., a salt of an iodide) and/or water thereto.

The concentration of the catalyst is, for example, about 5 to 10,000 ppm, preferably about 10 to 7,000 ppm, more preferably about 20 to 5,000 ppm (e.g., about 50 to 5,000 ppm), and in particular about 100 to 2,000 ppm on the basis of weight relative to the total amount of the liquid-phase system.

As the co-catalyst or accelerator constituting the catalytic system, there may be used, not being limited to the foregoing lithium iodide and methyl iodide, various alkali metal halides (e.g., a iodide such as potassium iodide or sodium iodide; a bromide such as lithium bromide, potassium bromide or sodium bromide), a hydrogen halide (e.g., hydrogen iodide, hydrogen bromide), an alkyl halide [an alkyl halide (a C₁₋₁₀alkyl halide, preferably a C₁₋₄alkyl halide) corresponding to a raw alcohol, for example a C₁₋₁₀alkyl iodide (e.g., a C₁₋₄alkyl iodide) such as methyl iodide, ethyl iodide or propyl iodide, a bromide corresponding to the alkyl iodide (e.g., methyl bromide, propyl bromide), or a chloride corresponding to the alkyl iodide (e.g., methyl chloride)]. Incidentally, the alkali metal halide (in particular a salt of an iodide) also functions as a stabilizer of a carbonylation catalyst (e.g., a rhodium catalyst). The co-catalyst(s) or accelerator(s) may be used singly or in combination. In particular, an alkali metal halide (in particular an alkali metal iodide) and an alkyl halide (in particular an alkyl iodide) are preferably used in combination.

The content of the co-catalyst or accelerator is about 0.1 to 40% by weight, preferably about 0.5 to 30% by weight, and more preferably about 1 to 25% by weight relative to total amount of the liquid-phase system. More specifically, in a production of a carboxylic acid by the foregoing carbonylation reaction of an alcohol, the content of the alkyl halide such as methyl iodide is about 0.1 to 30% by weight, preferably about 1 to 25% by weight, and more preferably about 5 to 20% by weight relative to the total amount of the liquid-phase system, and the content of the alkali metal halide such as lithium iodide is about 0.1 to 50% by weight, preferably about 0.5 to 40% by weight, and more preferably about 1 to 30% by weight relative to the total amount of the liquid-phase system.

Incidentally, in the reaction system a carboxylic acid ester (in particular an ester of a carboxylic acid with an alcohol, such as methyl acetate) may be included in a proportion of about 0.1 to 75% by weight, preferably about 0.2 to 50% by weight (e.g., about 0.2 to 25% by weight), and more preferably about 0.5 to 20% by weight (e.g., about 1 to 10% by weight) relative to the total amount of the liquid-phase system.

Carbon monoxide may be used as a pure gas, or may be used as a gas diluted with an inert gas (e.g., nitrogen, helium, carbon monoxide). The partial pressure of carbon monoxide in the reaction system may be selected suitably depending on the species of reaction, and others. For example, in a production of a carboxylic acid by a carbonylation reaction of an alcohol, the partial pressure of carbon monoxide in the reaction system is, for example, about 200 to 3,000 kPa, preferably about 400 to 2,000 kPa, and more preferably about 500 to 2,000 kPa as an absolute pressure.

Incidentally, carbon monoxide may be fed from the lower part of the reactor by sparging.

The reaction may be carried out in the presence or absence of a solvent, or carried out in the presence of a hydrogen gas.

Moreover, the reaction may be conducted in the presence of water. The existence of water in the reaction system is important because water acts on stability of a metal catalyst (such as a rhodium catalyst) and generating rate of an objective carboxylic acid (such as acetic acid). However, in the case where the proportion of water is too much in the reaction system, it is difficult to separate water efficiently in the purification step. Therefore, the proportion of water in the reaction system is usually not more than 20% by weight (e.g., about 0.001 to 20% by weight), preferably about 0.01 to 20% by weight, and more preferably about 0.1 to 15% by weight (e.g., about 1 to 15% by weight) as the water content in the liquid-phase system (or the reaction solution). In the case where the water content is too small, stability of the metal catalyst (such as a rhodium catalyst) and generating rate of the carboxylic acid (such as acetic acid) are deteriorated, and there is a possibility of significantly generating by-product (such as acetic anhydride). When the water content is over 20% by weight, the amount of water to be separated and recycled increases in the purification step, the vapor amount to be heated or the separation and removal equipment grow large, then too much costs are needed, as a result this is unfavorable industrially.

In the carbonylation reaction, the reaction temperature may for example be about 100 to 250° C. (preferably about 150 to 220° C., more preferably about 170 to 210° C.), and the reaction pressure (absolute pressure) may for example be about 1,000 to 5,000 kPa (e.g., about 1,500 to 4,000 kPa).

In the foregoing carbonylation reaction, a carboxylic acid having “n+1” carbon atoms (e.g., acetic acid) corresponding to an alcohol having “n” carbon atom(s) (e.g., methanol) is formed together with an ester of the formed carboxylic acid with the alcohol (e.g., methyl acetate), water generated with the esterification reaction, in addition an aldehyde having “n+1” carbon atoms (e.g., acetaldehyde) corresponding to the alcohol, a carboxylic acid having “n+2” carbon atoms (e.g., propionic acid), and others.

(Purification of Carboxylic Acid)

In the present invention, a carboxylic acid is purified from a carbonylation product by a separation step (A) of a higher bp catalyst component (or metal catalytic component), and a purification step (B) of a carboxylic acid.

(A) Separation Step of Higher Bp Catalyst Component

In a separation step of the higher bp catalyst component, the higher bp catalyst component (metal catalytic component, e.g., a carbonylation catalyst such as a rhodium catalyst, and an alkali metal halide) is separated from the reaction mixture obtained from the above-mentioned reaction system. The separation of the higher bp catalyst component may be conducted by a conventional separation method or separation apparatus, and may be usually carried out with the use of a distillation column (e.g., a plate column, a packed column, a flash distillation column). Moreover, the metal catalytic component may be separated by means of distillation in combination with a mist- or solid-collecting method which is widely used in industrial application.

The reaction mixture is separated into a vapor component as an overhead fraction containing the reaction product and a liquid component as a bottom fraction by distillation. In the separation step, the reaction mixture may be heated, or may be separated into the vapor component and the liquid component without heating. For example, when flash distillation is utilized, in adiabatic flash the reaction mixture may be separated into the vapor component and the liquid component with not heating but reduced pressure, and in thermostatic flash the reaction mixture may be separated into the vapor component and the liquid component with heating and reduced pressure. The reaction mixture may be separated into the vapor component and the liquid component by combining these flash conditions. The flash distillation step may be carried out at a temperature of about 80 to 200° C. under a pressure (absolute pressure) of about 50 to 1,000 kPa (e.g., about 100 to 1,000 kPa).

The separation step of the catalyst may be composed of a single step, or may be composed of a plurality of steps in combination. The bottom fraction (or metal catalytic fraction) separated by such a step is usually recycled to the reaction system.

Incidentally, the bottom fraction containing a higher bp catalyst component and a carboxylic acid having “n+2” carbon atoms may be separated from the reaction mixture to separate the bottom fraction into the higher bp catalyst component and the carboxylic acid having “n+2” carbon atoms.

(B) Purification Step

In the purification step, a carboxylic acid may be purified with the use of the higher bp component-separation column and the carboxylic acid-separating column. Moreover, a crude mixture containing an aldehyde may be fed to the higher bp component-separation column, or a crude mixture in which an aldehyde is separated (or removed) by the lower bp component-separation column beforehand may be fed to the higher bp component-separation column. The fraction (or overhead fraction) containing an aldehyde separated in a purification process of a carboxylic acid usually comprises useful components [e.g., an ester of a carboxylic acid having “n+1” carbon atoms with an alcohol having “n” carbon atom(s), an alkyl halide, water], and may be separated into the useful components and an aldehyde which deteriorates quality of an objective carboxylic acid by means of the following separation unit (aldehyde-separating column) for recycling the useful components to the reaction system.

In the purification step, for example, a carboxylic acid may be efficiently purified with the following systems: (b1) a system comprising a higher bp component-separation column, a carboxylic acid-separating column, and an aldehyde-separating column in that order; (b2) a system comprising a lower bp component-separation column, a higher bp component-separation column, and a carboxylic acid-separating column in that order; and (b3) a system comprising a lower bp component-separation column, a higher bp component-separation column, a carboxylic acid-separating column, and an aldehyde-separating column in that order. Incidentally, as the lower bp component-separation column, the higher bp component-separation column, the carboxylic acid-separating column, and the aldehyde-separating column, for example, there may be used conventional distillation columns such as a plate column, a packed column, and a flash distillation column.

The overhead fraction (crude mixture) in which the higher bp catalyst component is removed by the catalyst-separating column is usually composed mainly of an aldehyde having “n+1” carbon atoms, a carboxylic acid having “n+2” carbon atoms, a carboxylic acid having “n+1” carbon atoms, an ester of the carboxylic acid having “n+1” carbon atoms with an alcohol having “n” carbon atom(s), an alkyl halide, water, or others. If necessary, the aldehyde contained in the overhead fraction (crude mixture) may be fed to the lower bp component-separation column for separating from the crude mixture as an overhead fraction beforehand, or may be separated from the crude mixture in an appropriate step (an aldehyde-separating column).

(1) Lower Bp Component-Separation Column

The distillation temperature (or overhead temperature) and pressure (or overhead pressure) in the lower bp component-separation column may be selected depending on the species of aldehyde, an objective carboxylic acid and distillation column, and is not particularly limited as far as at least an aldehyde having “n+1” carbon atoms (preferably the aldehyde, and an alkyl halide such as methyl iodide) is separable by using difference between of an aldehyde to be separated as an overhead fraction and a bottom fraction in boiling point. For example, in the case carrying out purification of acetic acid by a plate column, the overhead pressure is about 10 to 1,000 kPa, preferably about 10 to 700 kPa, and more preferably about 50 to 500 kPa as an absolute pressure. In the case where the overhead pressure is too low, the boiling point of an aldehyde (in particular acetaldehyde in purification of acetic acid) becomes low, it is necessary to lower the temperature for condensing gaseous components, and as a result it is not preferred in cost. On the other hand, in the case where the overhead pressure is too high, the inner temperature of the column rises due to excessively added pressure, as a result there is a possibility that an aldehyde (in particular acetaldehyde) condensed within the column is polymerized within the column by exposing to high temperature.

Moreover, the overhead temperature may be adjusted by adjusting the overhead pressure, and may for example be about 20 to 180° C., preferably about 30 to 150° C., and more preferably about 40 to 120° C. As described above, in the lower bp component-separation column, an alkyl halide, a carboxylic acid ester, water or other components in addition to the aldehyde may be also separated as an overhead fraction by enhancing the overhead temperature. Such an overhead fraction may be fed to an aldehyde-separating column, and may be separated into an aldehyde and useful components (an alkyl halide, a carboxylic acid ester, and water). In such a case, the overhead temperature may be about 20 to 180° C., preferably about 30 to 150° C., and more preferably about 40 to 120° C.

Moreover, in the case of a plate column, the theoretical plate number is not particularly limited, and is about 5 to 30, preferably about 7 to 25, and more preferably about 8 to 20 depending on the species of component (or fraction) to be separated. Further, in order to highly (or accurately) separating an aldehyde by the fraction column, the theoretical plate number may be about 20 to 80, preferably about 25 to 60, and more preferably about 30 to 50. The removal of an aldehyde with a distillation column having such a plate number ensures significant decrease of load in a following distillation column.

In the lower bp component-separation column, the reflux ratio may for example be selected from about 0.5 to 3,000, and preferably about 1 to 2,000 depending on the above-mentioned theoretical plate number. As the theoretical plate number becomes larger, usually the reflux ratio may be smaller. Incidentally, the overhead fraction obtained by removing the bottom fraction in the separation step of the catalyst component is not necessarily subjected to reflux, and may be fed from the overhead of the lower bp component-separation column.

The bottom fraction separated by the lower bp component-separation column is usually composed mainly of a carboxylic acid having “n+2” carbon atoms, a carboxylic acid having “n+1” carbon atoms, an ester, an alkyl halide, water, and others.

(2) Higher Bp Component-Separation Column

In the higher bp component-separation column, noticing the viewpoint that a carboxylic acid having “n+1” carbon atoms and a carboxylic acid having “n+2” carbon atoms can be efficiently separated by utilizing difference between the both in boiling point, a carboxylic acid having “n+2” carbon atoms (e.g., propionic acid) is removed out of the system as a bottom fraction from the overhead fraction separated by the catalyst-separating column or the bottom fraction separated by the lower bp component-separation column. Therefore, propionic acid may be easily and accurately separated from acetic acid.

The distillation temperature and pressure in the higher bp component-separation column is not particularly limited as far as at least a carboxylic acid having “n+2” carbon atoms (e.g., propionic acid) is separable as a bottom fraction from an objective carboxylic acid (a carboxylic acid having “n+1” carbon atoms) by using difference between of the both in boiling point, and may be selected depending on the species of the foregoing carboxylic acid having “n+1” carbon atoms and carboxylic acid having “n+2” carbon atoms as well as that of distillation column.

For example, in the case purifying acetic acid as an objective carboxylic acid by a plate column, the overhead pressure is about 10 to 1,000 kPa, preferably about 10 to 700 kPa, and more preferably about 50 to 500 kPa as an absolute pressure. In the case where the overhead pressure is too low, the separation efficiency of the overhead fraction such as acetic acid, water, methyl iodide, and in some cases acetaldehyde becomes down, it is necessary to lower the temperature for condensing gaseous components efficiently, and as a result there is a possibility of cost thereof bringing disadvantages. On the other hand, in the case where the overhead pressure is too high, an excessive pressure is added to the column thereby increasing the bottom temperature, and further based on this, the pressure of vapor to be heated rises. As a result, equipment back up is required, and there is a possibility of cost thereof bringing disadvantages.

Moreover, the temperature of the column bottom may be adjusted by adjusting the overhead pressure. For example, in the case utilizing a plate column for purification of acetic acid, the temperature of the column bottom is not higher than 170° C. (e.g., about 50 to 170° C.), preferably about 70 to 170° C., more preferably about 100 to 170° C. Incidentally, in the case separating a carboxylic acid having “n+2” carbon atoms from the bottom fraction in which an aldehyde is separated by the lower bp component-separation column beforehand, the temperature of the column bottom of the higher bp component-separation column may for example be about 130 to 170° C., preferably about 140 to 170° C., and more preferably about 150 to 170° C. In the higher bp component-separation column, when the temperature of the column bottom is over 170° C., there is a possibility that acetic anhydride is generated by dehydration of acetic acid in the column bottom, and that the resultant acetic anhydride is distilled out from the overhead of the column to contaminate in acetic acid as a finished product.

In the case of the plate column, the theoretical plate number is not particularly limited, and is about 5 to 30, preferably about 7 to 25, and more preferably about 8 to 20 depending on the species of component to be separated.

Moreover, in the case of separating an aldehyde by the lower bp component-separation column beforehand, the theoretical plate number of the higher bp component-separation column may be about 7 to 30, preferably about 8 to 25, and more preferably about 10 to 20, and may be usually more than the theoretical plate number of the lower bp component-separation column. Incidentally, as described above, in the case where an aldehyde is highly separated beforehand with the use of the lower bp component-separation column having a large number of the theoretical plate, other lower-boiling impurities along with an aldehyde are also separated by the lower bp component-separation column, and as a result in the higher bp component-separation column, the overhead fraction and the bottom fraction can be accurately separated with the use of a distillation column whose theoretical plate number is less than that of the lower bp component-separation column. In such a case, the theoretical plate number of the higher bp component-separation column may for example be about 15 to 60, preferably about 15 to 50, and more preferably about 20 to 40.

In the higher bp component-separation column, the reflux ratio may for example be selected from about 0.5 to 10, and preferably about 0.7 to 5 depending on the theoretical plate number described above. The reflux ratio may be usually reduced by increasing the number of the theoretical plate. Incidentally, the overhead fraction obtained by removing the bottom fraction in the separation step of the catalytic fraction is not necessarily subjected to reflux, and may be fed from the overhead of the lower bp component-separation column.

Moreover, in the case separating an aldehyde by the lower bp component-separation column beforehand, the reflux ratio of the higher bp component-separation column may for example be about 0.1 to 10, and preferably about 0.5 to 5 (e.g., about 0.7 to 5) depending on the theoretical plate number described above.

The overhead fraction separated by the higher bp component-separation column is usually composed mainly of an aldehyde having “n+1” carbon atoms, a carboxylic acid having “n+1” carbon atoms, an ester, an alkyl halide, water, and others. Incidentally, in the case separating an aldehyde by the lower bp component-separation column beforehand, the overhead fraction mainly comprises the carboxylic acid having “n+1” carbon atoms, the ester, the alkyl halide, water, and others except the aldehyde.

Incidentally, when a salt of an iodide (e.g., a alkali metal iodide, an alkyl iodide) is use as a co-catalyst, hydrogen iodide is generated as reduction product by the action of water. Since the resultant hydrogen iodide produces azeotropic mixture having a maximum boiling point (127° C.) by an action of water thereby failing to separate from hydrous carboxylic acid (e.g., acetic acid), there is a possibility that hydrogen iodide is contaminated into acetic acid as a finished product. Therefore, in the higher bp component-separation column, along with adjusting the heating conditions (e.g., temperature, pressure), a condensed part of hydrogen iodide is formed in the column, a fraction which is eluted from the condensed part of hydrogen iodide by side cut and contains hydrogen iodide may be recycled to the reaction system; or a substrate alcohol (e.g., methanol) may be fed to the condensed part (or preferably a fraction containing a side cut hydrogen iodide) to convert hydrogen iodide to an alkyl iodide (e.g., methyl iodide), and then recycled to the reaction system. By such a method, higher quality acetic acid may be obtained.

(3) Carboxylic Acid-Separating Column

The overhead fraction which is separated by the higher bp component-separation column and contains a carboxylic acid having “n+1” carbon atoms usually comprises water (e.g., water generated by esterification), an alkyl halide, a carboxylic acid ester, and in some cases an aldehyde having “n+1” carbon atoms. Therefore, the alkyl halide and/or the carboxylic acid ester may be utilized as an azeotropic component of water, and water may be separated from the fraction containing a carboxylic acid having “n+1” carbon atoms efficiently by distilling in the presence of the ester and/or the alkyl halide as well as water.

The distillation temperature (or overhead temperature or bottom temperature) and pressure (or overhead pressure) in the carboxylic acid-separating column is not particularly limited as far as components such as water, an carboxylic acid ester, an alkyl halide, and in some cases an aldehyde are separable as an overhead fraction (or azeotropic component) from an objective carboxylic acid as a bottom fraction by utilizing difference between of the overhead fraction and the objective carboxylic acid in boiling point. The temperature and pressure may be selected depending on the species of overhead fraction and the objective carboxylic acid as well as distillation column. For example, in the case carrying out purification of acetic acid by a plate column, the overhead pressure may be about 10 to 1,000 kPa, preferably about 10 to 700 kPa, and more preferably about 50 to 500 kPa as an absolute pressure. In the case where the overhead pressure is too low, separation efficiency of the overhead fraction [water, methyl iodide, methyl acetate, and in some cases an aldehyde (particularly acetaldehyde in purification of acetic acid)] becomes low, it is necessary to lower the temperature for condensing gaseous components efficiently, and as a result it is not preferred in cost. On the other hand, in the case where the overhead pressure is too high, the inner temperature of the column rises due to excessively added pressure, and there is a possibility that an aldehyde (in particular acetaldehyde) which is condensed within the column is polymerized within the column by exposing to high temperature when an aldehyde is present into the column. Further, since the pressure of vapor to be heated rises, equipment back up is required, and there is a possibility of cost thereof bringing disadvantages.

The temperature of the column bottom may be adjusted by adjusting the overhead pressure. For example, in the case utilizing a plate column for purification of acetic acid, the temperature of the column bottom is not more than 170° C. (e.g., about 50 to 170° C.), preferably about 70 to 170° C., more preferably about 90 to 170° C. Moreover, in the case where an aldehyde is separated by the lower bp component-separation column beforehand, the bottom temperature of the carboxylic acid-separating column may for example be about 130 to 170° C., preferably about 140 to 170° C., and more preferably about 150 to 170° C. When the temperature of the column bottom of the carboxylic acid-separating column is over 170° C., there is a possibility that acetic anhydride is formed in the column bottom by dehydration of acetic acid, and that the resultant acetic anhydride is contaminated in acetic acid as a finished product.

In the case of a plate column, the theoretical plate number is not particularly limited, and is about 20 to 60, preferably about 25 to 55, and more preferably about 30 to 50 depending on the species of component (or fraction) to be separated, and may be usually more than the theoretical plate number of the higher bp component-separation column.

Moreover, in the case where an aldehyde is separated by the lower bp component-separation column beforehand, the theoretical plate number of the carboxylic acid-separating column is not particularly limited, and is about 10 to 80, preferably about 15 to 60 (e.g., about 15 to 50), and more preferably about 20 to 50 (e.g., about 30 to 50) depending on the species of component (or fraction) to be separated, and may be usually more than the theoretical plate number of the higher bp component-separation column. Moreover, in the case where an aldehyde is highly separated with the use of the lower bp component-separation column having a large number of the theoretical plate, other lower-boiling impurities along with an aldehyde are also separated by the lower bp component-separation column, as well as impurities are also separated by the higher bp component-separation column efficiently. Therefore, in the carboxylic acid-separating column, the overhead fraction and the bottom fraction may be accurately separated with the use of a distillation column whose theoretical plate number is less than that of the lower bp component-separation column and/or the higher bp component-separation column. In such a case, the theoretical plate number of the carboxylic acid-separating column may be about 7 to 50, preferably about 8 to 40, and more preferably about 10 to 30.

In the carboxylic acid-separating column, the reflux ratio may for example be selected from about 0.5 to 20, and preferably about 1 to 10 depending on the above-mentioned theoretical plate number. Moreover, in the case where an aldehyde is separated with the use of the lower bp component-separation column beforehand, the reflux ratio of the carboxylic acid-separating column may for example be about 1 to 100, and preferably about 1.5 to 80 depending on the theoretical plate number.

The overhead fraction separated from the carboxylic acid-separating column usually comprises a mainly aldehyde having “n+1” carbon atoms, in addition azeotropic components or useful components such as an ester, an alkyl halide, and water. The useful components may be separated from the aldehyde by a following separating unit (an aldehyde-separating column) to recycle to the reaction system. Moreover, in the case where the aldehyde in the lower bp component-separation column is separated beforehand, the above-mentioned overhead fraction mainly comprises useful components such as an ester, an alkyl halide and water, and may be recycled to the reaction system.

Incidentally, when hydrogen iodide is present in the carboxylic acid-separating column, in the carboxylic acid-separating column, along with adjusting the heating conditions (e.g., temperature, pressure), a condensed part of hydrogen iodide is formed in the column, a fraction which is eluted from the condensed part of hydrogen iodide by side cut and contains hydrogen iodide may be recycled to the reaction system; or a substrate alcohol (e.g., methanol) may be fed to the condensed part (or preferably a fraction containing a side cut hydrogen iodide) to convert hydrogen iodide into an alkyl iodide (e.g., methyl iodide), and then recycled to the reaction system.

Moreover, the objective carboxylic acid may be improved in purity by converting hydrogen iodide present in the carboxylic acid-separating column into an alkyl iodide (e.g., methyl iodide) or the like through feeding or infusing a substrate alcohol (e.g., methanol) or others in the column, and by separating the objective carboxylic acid from the converted product as a overhead fraction. The overhead fraction further containing useful components such as water may be recycled to the reactor.

Moreover, in order to improve a carboxylic acid (e.g., acetic acid) as a finished product in purity, the carboxylic acid as a finished product may be taken out from a site close to the column bottom of the carboxylic acid-separating column by side cut, or may be inhibited from contamination of a reducing substance (e.g., an aldehyde such as acetaldehyde or crotonaldehyde) by subjecting the reducing substance to ozone treatment. Further, after distillation off of the carboxylic acid as a finished product, impurities (e.g., an alkyl iodide such as hexyl iodide) may be removed by treating with an ion exchange resin exchanged with silver to improve the purity of the carboxylic acid.

Such a process insures production of higher quality acetic acid.

In the distillation step for separating the higher bp catalyst component, the lower bp component-separation column, the higher bp component-separation column, and the carboxylic acid-separating column, the overhead fraction may be fed to the subsequent step or subsequent separation column (or distillation column) in the form of gas, and may be usually fed to the subsequent step or subsequent separation column (or distillation column) in the form of liquid by condensation.

(4) Aldehyde-Separating Column

In the case feeding the overhead fraction which is separated by the catalyst-separating column and contains an aldehyde to the higher bp component-separation column, the overhead fraction separated from the carboxylic acid-separating column usually comprises, in addition to an aldehyde, water, an alkyl halide (methyl iodide), a carboxylic acid ester (methyl acetate), an objective carboxylic acid, and others. Therefore, the overhead fraction separated from the carboxylic acid-separating column may further be fed to an aldehyde-separating column to remove an aldehyde as an overhead fraction, and the resultant bottom fraction (containing water, an alkyl halide, a carboxylic acid ester, an objective carboxylic acid) may be recycled to the reaction system. Incidentally, since the aldehyde (e.g., acetaldehyde) has higher vapor pressure compared with other impurities, it is possible to separate the aldehyde easily by the aldehyde-separating column.

Moreover, when an aldehyde is highly separated in the lower bp component-separation column beforehand, the overhead fraction separated from the lower bp component-separation column usually comprises, in addition to an aldehyde (e.g., acetaldehyde), an alkyl halide (e.g., methyl iodide), water, an carboxylic acid ester (e.g., methyl acetate), and others. In such a case, the aldehyde may be further fed to the aldehyde-separating column to be removed as an overhead fraction, and the resultant bottom fraction (containing an alkyl halide, water, a carboxylic acid ester, an objective carboxylic acid) may be recycled to the reaction system.

The temperature (overhead temperature) and pressure (overhead pressure) in the aldehyde-separating column may be selected depending on the species of aldehyde and alkyl halide as well as distillation column, and is not particularly limited as far as at least an aldehyde (e.g., acetaldehyde) is separable as an overhead fraction from the overhead fraction obtained in the higher bp component-separation column by utilizing difference between the aldehyde and other components (particularly an alkyl halide) in boiling point. For example, in the case using a plate column as the aldehyde-separating column for purification of acetic acid, the overhead pressure is about 10 to 1,000 kPa, preferably about 10 to 700 kPa, and more preferably about 10 to 500 kPa as an absolute pressure. In the case where the overhead pressure is too low, separation efficiency of acetaldehyde becomes low, it is necessary to lower the temperature for condensing gaseous components efficiently, and as a result it is not preferred in cost. On the other hand, in the case where the overhead pressure is too high, the inner temperature of the column rises due to excessively added pressure, as a result there is a possibility that acetaldehyde which is condensed within the column is polymerized within the column by exposing to high temperature thereby being contaminated in the bottom fraction.

Moreover, the overhead temperature may be adjusted by adjusting the overhead pressure, and for example, is about 10 to 80° C., preferably about 20 to 70° C., and more preferably about 40 to 60° C.

In the case where the aldehyde-separating column is a plate column, the theoretical plate number may be usually more than the theoretical plate number of the higher bp component-separation column, or may for example be about 5 to 40, preferably about 8 to 35, and more preferably about 10 to 30 depending on the species of component (or fraction) to be separated. Moreover, in the case where the overhead fraction which is separated by the lower bp component-separation column and contains an aldehyde is fed to the aldehyde-separating column, the theoretical plate number of the aldehyde-separating column may be, in a plate column, usually more than the theoretical plate number of the lower bp component-separation column, and may for example be selected from about 10 to 80, preferably about 20 to 60, and more preferably about 30 to 50 depending on the species of component (or fraction) to be separated.

In the aldehyde-separating column, the reflux ratio may be selected from about 1 to 1,000, preferably about 10 to 800, and preferably about 50 to 600 (e.g., about 100 to 600) depending on the above-mentioned theoretical plate number.

Incidentally, when the overhead fraction (or crude mixture) which is separated by the catalyst-separating column and contains an aldehyde is fed to the higher bp component-separation column, hydrogen iodide exists in the aldehyde-separating column in some cases. In such a case, hydrogen iodide present in the aldehyde-separating column may be converted into an alkyl iodide (e.g., methyl iodide) through feeding or infusing a substrate alcohol (e.g., methanol) or others in the aldehyde-separating column to separate as a bottom fraction in the column for recycling to the reaction system.

In the present invention, such a separation and purification process insures larger energy efficiency, and significant reduction of a vapor amount to be used per 1,000 g of a carboxylic acid compared with a conventional purification process. For example, the vapor amount to be required for heating in purification of acetic acid [e.g., (1) a higher bp component-separation column, a carboxylic acid-separating column, and an aldehyde-separating column, (2) a lower bp component-separation column, a higher bp component-separation column, and a carboxylic acid-separating column, (3) a lower bp component-separation column, a higher bp component-separation column, a carboxylic acid-separating column, and an aldehyde-separating column] is about 500 to 2,000 g, preferably about 500 to 1,500 g, and more preferably about 600 to 1,000 g relative to 1,000 g of acetic acid.

According to the present invention, at least a carboxylic acid having “n+2” carbon atoms is removed from a reaction mixture formed by a carbonylation reaction, and then the distillation can be carried out in the presence of at least water and an ester of a carboxylic acid with an alcohol, wherein water and ester are generated in the reaction system. Accordingly, impurities are efficiently separated from the reaction mixture to produce a carboxylic acid (in particular acetic acid) easily and efficiently. Moreover, a purified carboxylic acid may be produced with removing water without circulating an excess amount of a carboxylic acid (in particular acetic acid) through the system. Further, since an ester and water formed in the reaction system can be utilized as azeotropic components, a carboxylic acid (in particular acetic acid) can be highly purified without adding an azeotropic component, and therefore, a highly purified carboxylic acid (in particular acetic acid) can be produced at high energy efficiency.

### INDUSTRIAL APPLICABILITY

According to the present invention, in a series of steps as described above, particularly in a carboxylic acid-separating column, since a carboxylic acid ester or an alkyl halide (such as methyl iodide) capable of azeotrope with water may coexist with water, water may be removed efficiently without circulating an excess amount of a carboxylic acid through the reaction system. Moreover, an aldehyde may be efficiently removed with the use of a lower bp component-separation column or aldehyde-separating column. Therefore, a carboxylic acid (e.g., acetic acid) may be highly purified at high-energy efficiency and a low cost, and both of energy cost and equipment expenses may be reduced. Accordingly, the present invention is useful for industrial production of a carboxylic acid.

### EXAMPLES

The following examples are intended to describe this invention in further detail and should by no means be interpreted as defining the scope of the invention. Incidentally, in Examples, pressure is shown in absolute pressure.

### Example 1

(1) Carbonylation Reaction

A rhodium catalyst, lithium iodide, methyl iodide, and water were supplied to a reactor at prescribed amounts so that the concentration of the rhodium catalyst, that of lithium iodide, that of methyl iodide, and that of water were 400 ppm, 0.5 mol/L, 14% by weight, and 8% by weight in a mixture (liquid-phase system), respectively. The reaction was carried out at 187° C. with feeding carbon monoxide and methanol to the reactor continuously to form acetic acid.

(2) Separation Step of Higher Bp Catalyst Component

The reaction mixture (or crude reaction solution) obtained in the reaction step (1) was distilled with the use of a distillation column (catalyst-separating column) (temperature of 132° C., pressure of 252 kPa), and was separated into a less-volatile phase (bottom fraction) and a higher-volatile phase (overhead fraction). The less-volatile phase containing the rhodium catalyst and the salt of iodide (lithium iodide) as main components, and small amounts of methyl iodide, water and acetic acid was sent back to the reaction step from the bottom of the catalyst-separating column. On the other hand, the higher-volatile phase containing, along with acetic acid, methyl acetate, methyl iodide and water was distilled off as a distillate from the overhead of the catalyst-separating column. The distillate contained 33.77% by weight of methyl iodide, 3.58% by weight of methyl acetate, 7.60% by weight of water, 0.01% by weight of propionic acid, 0.01% by weight of acetaldehyde, and acetic acid as the rest.

(3) Purification Step

The overhead fraction (crude mixture) distilled from the overhead in the separation step (2) of the higher bp catalyst component was fed to the overhead of a distillation column (higher bp component-separation column) (theoretical plate number of 12, operation pressure of 196 kPa as overhead pressure) at a rate of 1,200 g/h. Incidentally, the reflux of the higher bp component-separation column was not necessary because the above fraction (distillate) was fed to the overhead of the column. The bottom solution was withdrawn from the bottom of the column at a bottom rate of 0.7 g/h. The bottom solution contained 2.56% by weight of propionic acid, and acetic acid as the rest.

The overhead fraction distilled from the overhead of the higher bp component-separation column was supplied to the 17th plate from the top of a distillation column (carboxylic acid-separating column) (theoretical plate number of 38, operation pressure of 98 kPa as overhead pressure) at a rate of 1199.3 g/h. The reflux ratio of the carboxylic acid-separating column was 2.2, and acetic acid as a finished product was obtained from the bottom of the column at a bottom rate of 625 g/h. The bottom solution obtained from the column bottom contained 300 ppm of water, 160 ppm of propionic acid, and acetic acid as the rest.

The overhead fraction distilled from the overhead of the carboxylic acid-separating column was supplied to the 9th plate from the top of an aldehyde-separating column (theoretical plate number of 18, operation pressure of 196 kPa as overhead pressure) at a rate of 574.3 g/h. The reflux ratio of the aldehyde-separating column was 200, and the bottom solution was obtained from the bottom of the column at a bottom rate of 573.3 g/h. The bottom solution from the column bottom contained 70.5% by weight of methyl iodide, 7.5% by weight of methyl acetate, 16% by weight of water, and acetic acid as the rest.

The vapor amount to be used for heating from the higher bp component-separation column to the aldehyde-separating column was 744 g relative to 1,000 g of acetic acid as a finished product.

### Example 2

(1) Purification Step

The overhead fraction (crude mixture) which was obtained in the separation step (2) of the higher bp catalyst component in Example 1 was supplied to the 9th plate from the top of a first distillation column (lower bp component-separation column) (theoretical plate number of 10, operation pressure of 294 kPa as overhead pressure) at a rate of 1,200 g/h. The reflux ratio of the lower bp component-separation column was 1592, and the distillate was distilled off from the overhead of the column at a distillation rate of 0.6 g/h. The resultant overhead fraction contained 20% by weight of acetaldehyde, 3% by weight of water, and methyl iodide as the rest.

The bottom solution which was withdrawn from the bottom of the lower bp component-separation column was fed to the overhead of a second distillation column (higher bp component-separation column) (theoretical plate number of 14, operation pressure in the distillation column of 101 kPa as overhead pressure) at a rate of 119.4 g/h. Incidentally, the reflux of the higher bp component-separation column was not necessary because the bottom solution was fed to the overhead of the column. The bottom solution was withdrawn from the column bottom of the higher bp component-separation column at a bottom rate of 0.6 g/h. The bottom solution from the column bottom contained 4.6% by weight of propionic acid, and acetic acid as the rest.

The overhead fraction distilled from the overhead of the higher bp component-separation column was supplied to the 15th plate from the top of a third distillation column (carboxylic acid-separating column) (theoretical plate number of 40, operation pressure of 101 kPa as overhead pressure) at a rate of 1198.8 g/h. The reflux ratio of the carboxylic acid-separating column was 2.09, and acetic acid as a finished product was obtained from the bottom of the column at a bottom rate of 625 g/h. The obtained bottom solution contained 300 ppm of water, 148 ppm of propionic acid, and acetic acid as the rest.

In the carboxylic acid-separating column, the overhead fraction distilled from the overhead of the column contained 70.5% by weight of methyl iodide, 7.5% by weight of methyl acetate, 16% by weight of water, and acetic acid as the rest.

The vapor amount to be used for heating from the lower bp component-separation column to the carboxylic acid-separating column was 884 g relative to 1,000 g of acetic acid as a finished product.

### Example 3

(1) Purification Step

The overhead fraction (crude mixture) distilled from the overhead in the separation step (2) of the higher bp catalyst component in Example 1 was supplied to the 22nd plate from the top of a first distillation column (lower bp component-separation column) (theoretical plate number of 40, operation pressure of 101 kPa as overhead pressure) at a rate of 1200 g/h. The reflux ratio of the lower bp component-separation column was 1.37, and the bottom solution was withdrawn from the column bottom at a bottom rate of 631.1 g/h. The bottom solution contained 0.9% by weight of water, 0.02% by weight of propionic acid, and acetic acid as the rest.

The bottom solution which was withdrawn from the column bottom of the lower bp component-separation column was supplied to the second plate from the top of a second distillation column (higher bp component-separation column) (theoretical plate number of 27, operation pressure of 98 kPa as overhead pressure) at a rate of 631.1 g/h. The reflux ratio of the higher bp component-separation column was 1, and the bottom solution was withdrawn from the column bottom at a bottom rate of 0.3 g/h. The bottom solution from the column bottom contained 2.1% by weight of propionic acid, and acetic acid as the rest.

The distillate from the overhead of the higher bp component-separation column was supplied to the 12th plate from the top of a third distillation column (carboxylic acid-separating column) (theoretical plate number of 20, operation pressure of 98 kPa as overhead pressure) at a rate of 630.8 g/h. The reflux ratio of the carboxylic acid-separating column was 62.4, and acetic acid as a finished product was obtained from the column bottom at a bottom rate of 625 g/h. The obtained bottom solution contained 300 ppm of water, 152 ppm of propionic acid, and acetic acid as the rest.

The overhead fraction distilled from the overhead of the lower bp component-separation column was further supplied to the 40th plate from the top of a forth distillation column (aldehyde-separating column) (theoretical plate number of 40, operation pressure of 196 kPa as overhead pressure) at a rate of 568.9 g/h. The reflux ratio of the aldehyde-separating column was 400, and the bottom solution was obtained from the column bottom at a bottom rate of 568.3 g/h. The obtained bottom solution contained 71.5% by weight of methyl iodide, 7.6% by weight of methyl acetate, 16% by weight of water, and acetic acid as the rest.

The vapor amount to be used for heating from the first distillation column (lower bp component-separation column) to the forth distillation column (aldehyde-separating column) was 1078 g relative to 1000 g of acetic acid as a finished product.

Assuming that the equipment expenses of Example 2 were 1, those of Example 3 were 3.8.

### Comparative Example 1

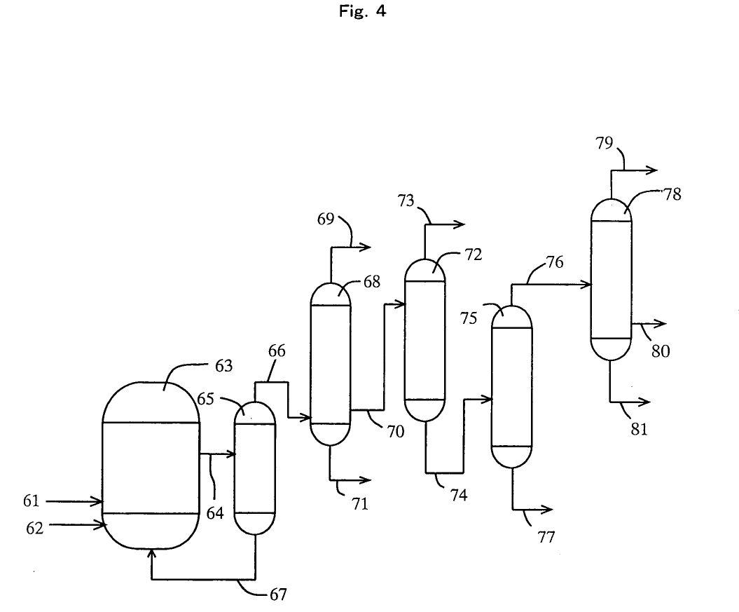

Based on the flow diagram shown in FIG. 4, acetic acid was purified.

(1) Carbonylation Reaction

A rhodium catalyst, lithium iodide, methyl iodide, and water were supplied to a reactor 63 at prescribed amounts so that the concentration of the rhodium catalyst, that of lithium iodide, that of methyl iodide, and that of water were 400 ppm, 0.5 mol/L, 14% by weight, and 8% by weight in a mixture (liquid-phase system), respectively. The reaction was carried out at 187° C. with feeding carbon monoxide and methanol through feed lines 61 and 62, respectively, to the reactor 63 continuously to produce acetic acid.

(2) Separation Step of Higher Bp Catalyst Component

The reaction mixture (or crude reaction solution) obtained in the carbonylation reaction (1) was fed to a distillation column (catalyst-separating column) 65 (temperature of 132° C., pressure of 252 kPa) through a feed line 64, and was separated into a less-volatile phase (bottom fraction) and a higher-volatile phase (overhead fraction). The less-volatile phase containing the rhodium catalyst and the salt of iodide (lithium iodide) as main components, and small amounts of methyl iodide, water and acetic acid was sent back to the reaction system 63 through a recycle line 67 from the bottom of the catalyst-separating column. On the other hand, the higher-volatile phase containing, along with acetic acid, methyl acetate, methyl iodide and water was distilled off as a distillate from the overhead of the catalyst-separating column. The distillate contained 33.77% by weight of methyl iodide, 3.58% by weight of methyl acetate, 7.60% by weight of water, 0.01% by weight of propionic acid, 0.01% by weight of acetaldehyde, and acetic acid as the rest.

(3) Purification Step

The overhead fraction (crude mixture) distilled from the overhead in the separation step (2) of the higher bp catalyst component was supplied to the 12th plate from the top of a first distillation column 68 (theoretical plate number of 12, operation pressure of 235.2 kpa as overhead pressure) through a feed line 66 at a rate of 1200 g/h. The reflux ratio of the higher bp component-separation column 68 was 0.87, the bottom solution was withdrawn from the column bottom through a bottom line 71 at a bottom rate of 12 g/h, and the overhead fraction was removed from the overhead through a distillation line 69. Moreover, a side-cut solution was withdrawn from the 10th plate from the top of the first distillation column at a discharge amount of 667 g/h. The bottom solution contained 0.02% by weight of methyl acetate, 1.64% by weight of water, 0.05% by weight of propionic acid, and acetic acid as the rest. The side-cut solution contained 1.3% by weight of methyl iodide, 4.9% by weight of water, 0.017% by weight of propionic acid, and acetic acid as the rest.

The side-cut solution of the first distillation column was supplied to the third plate from the top of a second distillation column 72 (theoretical plate number of 19, operation pressure of 274.4 kPa as overhead pressure) through a feed line 70 at a rate of 667 g/h. The reflux ratio of the second distillation column 72 was 8, the overhead fraction was separated through a distillation line 73 from the overhead of the column, and the bottom solution was obtained from the column bottom at a bottom rate of 600 g/h. The obtained bottom solution contained 0.6% by weight of water, 0.017% by weight of propionic acid, and acetic acid as the rest.

The bottom solution obtained from the column bottom of the second distillation column was supplied to the 7th plate from the top of a third distillation column 75 (theoretical plate number of 16, operation pressure of 215.6 kPa as overhead pressure) through a feed line 74 at a rate of 600 g/h. The reflux ratio of the third distillation column 75 was 5, the bottom fraction was separated from the column bottom through a bottom line 77, and the distillate was obtained from the overhead of the column at a distillation rate of 599.46 g/h. The obtained distillate contained 0.6% by weight of water, 0.015% by weight of propionic acid, and acetic acid as the rest.

The overhead fraction distilled from the overhead of the third distillation column was supplied to the 12th plate from the top of a forth distillation column 78 (theoretical plate number of 22, operation pressure of 98 kPa as overhead pressure) through a feed line 76 at a rate of 599.46 g/h. The reflux ratio of the forth distillation column 78 was 45. Along with the distillate was distilled off from the overhead of the column through a distillation line 79 at a distillation rate of 4.4 g/h, a side-cut solution was withdrawn from the 22nd plate from the top of the distillation column through an extraction line 80 at an extraction rate of 595 g/h to obtain acetic acid as a finished product. Moreover, the bottom fraction obtained from the column bottom was removed through a bottom line 81. The distillate obtained from the overhead of the column contained 78.4% by weight of water, and acetic acid the rest. Moreover, the side-cut solution contained 300 ppm of water, 151 ppm of propionic acid, and acetic acid as the rest.

The vapor amount to be used for heating from the first distillation column to the forth distillation column was 3296 g relative to 1000 g of acetic acid as a finished product.

Assuming that the equipment expenses of Example 1 were 1, those of Comparative Example 1 were 2.2. Moreover, assuming that the equipment expenses of Example 2 were 1, those of Comparative Example 1 were 8.3.

### Comparative Example 2

(1) Purification Step

The overhead fraction (crude mixture) distilled from the overhead in the separation step (2) of the higher bp catalyst component in Comparative Example 1 was supplied to the 20th plate from the top of a first distillation column (theoretical plate number of 20, operation pressure of 235.2 kPa as overhead pressure) at a rate of 1200 g/h. The reflux ratio of the first distillation column was 0.65, and the bottom solution was withdrawn from the column bottom at a bottom rate of 6 g/h. Moreover, a side-cut solution was withdrawn from the 19th plate from the top of the distillation column at a discharge amount of 667 g/h. The bottom solution contained 0.01% by weight of methyl iodide, 0.02% by weight of methyl acetate, 1.7% by weight of water, 0.04% by weight of propionic acid, and acetic acid as the rest. The side-cut solution contained 1.5% by weight of methyl iodide, 3.6% by weight of water, 0.018% by weight of propionic acid, and acetic acid as the rest.

The side-cut solution of the first distillation column was supplied to the third plate from the top of a second distillation column (theoretical plate number of 42, operation pressure of 176 kPa as overhead pressure) at a rate of 667 g/h. The reflux ratio of the second distillation column was 7, and the bottom solution was obtained from the column bottom at a bottom rate of 600 g/h. The bottom solution contained 0.29% by weight of water, 0.016% by weight of propionic acid, and acetic acid as the rest.

The bottom solution obtained from the column bottom of the second distillation column was supplied to the 17th plate from the top of a third distillation column (theoretical plate number of 30, operation pressure of 215.6 kPa as overhead pressure) at a rate of 600 g/h. The reflux ratio of the third distillation column was 5, and the distillate was obtained from the overhead of the column at a distillation rate of 599.46 g/h. The distillate contained 0.3% by weight of water, 0.018% by weight of propionic acid, and acetic acid as the rest.

The overhead fraction distilled from the overhead of the third distillation column was supplied to the second plate from the top of a forth distillation column (theoretical plate number of 22, operation pressure of 98 kPa as overhead pressure) at a rate of 599.46 g/h. The reflux ratio of the forth distillation column was 66, and the distillate was obtained from the overhead of the column at a distillation rate of 4.4 g/h. Moreover, a side-cut solution was withdrawn from the 22nd plate from the top of the forth distillation column at a discharge amount of 595 g/h to obtain acetic acid as a finished product. The distillate from the overhead of the column contained 37.0% by weight of water, and acetic acid as the rest. The side-cut solution contained 300 ppm of water, 285 ppm of propionic acid, and acetic acid as the rest.

The vapor amount to be used for heating from the first distillation column to the forth distillation column was 2215 g relative to 1000 g of acetic acid as a finished product.

Assuming that the equipment expenses of Example 1 were 1, those of Comparative Example 2 were 1.6. Moreover, assuming that the equipment expenses of Example 2 were 1, those of Comparative Example 2 were 5.9.

1. A process for producing a carboxylic acid comprising
allowing an alcohol having “ⁿ” carbon atom(s) or a derivative thereof to react with carbon monoxide continuously in the presence of a catalytic system, and
purifying the resultant reaction mixture to give a purified carboxylic acid having “n+1” carbon atoms,
wherein a higher bp catalyst component is separated from the reaction mixture to give a crude mixture containing at least a carboxylic acid having “n+2” carbon atoms, a carboxylic acid having “n+1” carbon atoms, an ester of the carboxylic acid having “n+1” carbon atoms with the alcohol, and water;
the crude mixture is fed to a higher bp component-separation column, and is separated into a bottom fraction and an overhead fraction, the bottom fraction contains at least the carboxylic acid having “n+2” carbon atoms, and the overhead fraction contains at least the carboxylic acid having “n+1” carbon atoms, the ester of the carboxylic acid having “n+1” carbon atoms with the alcohol, and water; and
the overhead fraction from the higher bp component-separation column is separated by a carboxylic acid-separating column into a bottom fraction and an overhead fraction, the bottom fraction contains the carboxylic acid having “n+1” carbon atoms, and the overhead fraction contains at least the ester and water.

2. A process according to claim 1, wherein the reaction mixture contains water in a proportion of not more than 20% by weight.

3. A process according to claim 1, wherein the crude mixture further contains an aldehyde having “n+1” carbon atoms, and the crude mixture is fed to the higher bp component-separation column.

4. A process according to claim 1, wherein the crude mixture containing the carboxylic acid having “n+2” carbon atoms, an aldehyde having “n+1” carbon atoms, the carboxylic acid having “n+1” carbon atoms, the ester of the carboxylic acid having “n+1” carbon atoms with the alcohol and water is fed to the higher bp component-separation column, and is separated into the bottom fraction and the overhead fraction, the bottom fraction contains the carboxylic acid having “n+2” carbon atoms, and the overhead fraction contains the aldehyde having “n+1” carbon atoms, the carboxylic acid having “n+1” carbon atoms, the ester of the carboxylic acid having “n+1” carbon atoms with the alcohol, and water;
the overhead fraction from the higher bp component-separation column is separated by the carboxylic acid-separating column into the bottom fraction and the overhead fraction, the bottom fraction contains the carboxylic acid having “n+1” carbon atoms, and the overhead fraction contains at least the aldehyde, the ester and water;
the overhead fraction from the carboxylic acid-separating column is separated by an aldehyde-separating column into an overhead fraction and a bottom fraction, the overhead fraction contains the aldehyde, and the bottom fraction contains at least the ester and water; and
the bottom fraction from the aldehyde-separating column is recycled to the reaction system.

5. A process according to claim 4, wherein the catalytic system comprises a catalyst containing a metal element of the Group 8 of the Periodic Table of Elements, an alkali metal halide, and an alkyl halide;
distillation in the carboxylic acid-separating column is carried out in the presence of the ester of the carboxylic acid having “n+1” carbon atoms with the alcohol, the alkyl halide and water for separating the bottom fraction from the overhead fraction, the bottom fraction contains the carboxylic acid having “n+1” carbon atoms, and the overhead fraction contains water, the alkyl halide and the ester;
the overhead fraction from the carboxylic acid-separating column is separated by the aldehyde-separating column into the overhead fraction and the bottom fraction, the overhead fraction contains the aldehyde, and the bottom fraction contains water, the alkyl halide and the ester; and
the bottom fraction from the aldehyde-separating column is recycled to the reaction system.

6. A process according to claim 1, wherein the crude mixture in which at least an aldehyde having “n+1” carbon atoms has been removed is fed to the higher bp component-separation column.

7. A process according to claim 1, wherein the higher bp catalyst component is separated from the reaction mixture to give a crude mixture, and the resultant crude mixture is fed to a lower bp component-separation column, and is separated into the overhead fraction and the bottom fraction, the overhead fraction contains at least an aldehyde having “n” carbon atom(s), and the bottom fraction contains at least the carboxylic acid having “n+2” carbon atoms;
the bottom fraction from the lower bp component-separation column is separated by the higher bp component-separation column into the bottom fraction and the overhead fraction, the bottom fraction contains the carboxylic acid having “n+2” carbon atoms, and the overhead fraction contains at least the carboxylic acid having “n+1” carbon atoms, the ester of the carboxylic acid having “n+1” carbon atoms with the alcohol, and water; and
the overhead fraction from the higher bp component-separation column is separated by the carboxylic acid-separating column into the bottom fraction containing the carboxylic acid having “n+1” carbon atoms and the overhead fraction containing at least the ester and water.

8. A process according to claim 7, wherein the catalytic system comprises a catalyst containing a metal element of the Group 8 of the Periodic Table of Elements, an alkali metal halide, and an alkyl halide; and
distillation in the carboxylic acid-separating column is carried out in the presence of the ester, the alkyl halide and water to give the bottom fraction containing the carboxylic acid having “n+1” carbon atoms, and the overhead fraction containing at least the ester, the alkyl halide and water.

9. A process according to claim 7 or 8, wherein the overhead fraction separated by the carboxylic acid-separating column is recycled to the reaction system.

10. A process according to claim 7, wherein the overhead fraction separated by the lower bp component-separation column is further fed to an aldehyde-separating column to separate an overhead fraction containing an aldehyde having “n+1” carbon atoms to give a bottom fraction containing at least the ester and water; and
the bottom fraction is recycled to the reaction system.

11. A process according to any one of claims 1, 4 and 7, wherein distillation in the carboxylic acid-separating column is carried out in the presence of at least the ester and water to give the bottom fraction containing the carboxylic acid having “n+1” carboxylic acid, and the overhead fraction.

12. A process according to claim 1 which comprises
allowing at least one member selected from the group consisting of methanol, methyl acetate and dimethyl ether to react with carbon monoxide continuously in the presence of the catalytic system, and
purifying the resultant reaction mixture to produce a purified acetic acid,
wherein the higher bp catalyst component is separated from the reaction mixture to give the crude mixture;
the crude mixture is fed to the higher bp component-separation column, and is separated into the bottom fraction and the overhead fraction, the bottom fraction contains at least propionic acid, and the overhead fraction contains at least acetic acid, methyl acetate and water; and
the overhead fraction from the higher bp component-separation column is separated by the carboxylic acid-separating column into the bottom fraction and the overhead fraction, the bottom fraction contains said acetic acid, and the overhead fraction contains at least said methyl acetate and water.

13. A process according to claim 12, wherein the catalytic system comprises a catalyst containing a rhodium catalyst, an alkali metal iodide and methyl iodide;
the crude mixture is separated by the higher bp component-separation column into the bottom fraction and the overhead fraction, the bottom fraction contains at least propionic acid, and the overhead fraction contains acetic acid, methyl acetate, methyl iodide and water; and
the overhead fraction from the higher bp component-separation column is distilled by the carboxylic acid-separating column in the presence of said methyl acetate and methyl iodide, and is separated into the bottom fraction and the overhead fraction, the bottom fraction contains said acetic acid, and the overhead fraction contains said methyl acetate, methyl iodide and water.

14. A system for producing a carboxylic acid which comprises
a reaction system for allowing an alcohol having “n” carbon atom(s) or a derivative thereof to react with carbon monoxide continuously in the presence of a catalytic system,
a catalyst-separating column for separating a higher bp catalyst component from a reaction mixture generated in the reaction system,
a higher bp component-separation column for separating a crude mixture obtained by a separation in the catalyst-separating column and containing at least a carboxylic acid having “n+2” carbon atoms, a carboxylic acid having “n+1” carbon atoms, an ester of the carboxylic acid having “n+1” carbon atoms with the alcohol, and water, into a bottom fraction and an overhead fraction, wherein the bottom fraction contains at least the carboxylic acid having “n+2” carbon atoms, and the overhead fraction contains at least the carboxylic acid having “n+1” carbon atoms, the ester of the carboxylic acid having “n+1” carbon atoms with the alcohol, and water, and
a carboxylic acid-separating column for separating the overhead fraction separated by the higher bp component-separation column into a bottom fraction and an overhead fraction, wherein the bottom fraction contains the carboxylic acid having “n+1” carbon atoms, and the overhead fraction contains at least the ester and water.
